(19) United States
(12) Patent Application Publication
(10) Pub. No.: US 2026/0115225 A1
(43) Pub. Date: Apr. 30, 2026

(54) Effervescent Laxative Composition

(71) Applicant: L. PERRIGO COMPANY, Allegan, MI (US)

(72) Inventors: Lisa K. LUPTON, Kalamazoo, MI (US); Kiran Kumar MUPPIREDDY, Portage, MI (US); Inderdeep BHATIA, Portage, MI (US); Carlos O. PAZ, Fairview, NJ (US); Amit BANSAL, Portage, MI (US)

(21) Appl. No.: 19/344,840

(22) Filed: Sep. 30, 2025

Related U.S. Application Data
(60) Provisional application No. 63/702,362, filed on Oct. 2, 2024.

(51) Int. Cl. A61K 31/765; A61J 1/14; A61K 47/02; A61K 47/12; A61K 47/26

(52) U.S. Cl. CPC A61K 31/765; A61J 1/1431; A61K 47/02; A61K 47/12; A61K 47/26

(57) ABSTRACT

An effervescent laxative composition comprising an acid component, an alkaline component, and a polyethylene glycol is disclosed.

### CROSS-REFERENCE TO RELATED APPLICATIONS

This application claims priority to and benefit of U.S. provisional application No. 63/702,362, filed Oct. 2, 2024, the entire contents of which are incorporated herein by reference.

### TECHNICAL FIELD

The present disclosure relates to compositions comprising polyethylene glycol and an effervescent additive.

### BACKGROUND

Polyethylene glycol (PEG), particularly PEG 3350, is a known osmotic laxative. Typically, PEG laxative compositions are sold as dry compositions which are to be combined with an aqueous medium to provide a consumable laxative solution. Examples of such products include Miralax® (Bayer and Gavilax® (Gavis Pharmaceuticals, NJ). Effervescent PEG compositions are also known in the art, for example in U.S. Pat. No. 6,444,198. There remains a need in the art for improved PEG and effervescent PEG compositions.

### SUMMARY

The present disclosure includes a dry PEG composition comprising an effervescent agent that does not adversely affect the stability of the PEG. In particular, the composition remains shelf-stable in a multi-dose container and when stored for extended periods of time. In practice, this allows a dry PEG composition to be mixed and packaged with a proton source (acid) and a carbonate or bicarbonate base (base). The stability of the compositions of the present disclosure helps mitigate, reduce, or eliminate issues with agglomeration, dispersibility, water content, and chemical instability (e.g., excessive formaldehyde and total aldehyde formation). The present disclosure also provides multiple servings of a packaged PEG product comprising the effervescent agent. Accordingly, the present disclosure enables a consistent consumer experience from serving to serving.

In some aspects, the present disclosure provides a powdered effervescent laxative composition, the powdered composition comprising: about 70 weight percent or more of polyethylene glycol having a weight average molecular weight of between about 3,000 and about 4,000 g/mol; an effervescent system including (i) an acid component selected from the group consisting of monosodium citrate, disodium citrate, trisodium citrate, maleic acid, fumaric acid, adipic acid, and combinations thereof and (ii) an alkaline component selected from the group consisting of sodium bicarbonate, sodium carbonate, potassium bicarbonate, potassium carbonate, calcium bicarbonate, calcium carbonate, and combinations thereof; and wherein the effervescent system has a molar ratio of (i) the acid component to (ii) the alkaline component of from about 1:1 to about 1:4.

In some aspects, the present disclosure provides a packaged and shelf-stable powdered effervescent laxative composition, the packaged composition comprising: a sealed container; a powdered effervescent laxative composition contained in the sealed container; wherein the powdered effervescent laxative composition includes about 70 weight percent or more of polyethylene glycol having a weight average molecular weight of from 3000 to 4000 g/mol; an effervescent system including (i) an acid component selected from the group consisting of monosodium citrate, disodium citrate, trisodium citrate, maleic acid, fumaric acid, adipic acid, and combinations thereof and (ii) an alkaline component selected from the group consisting of sodium bicarbonate, sodium carbonate, potassium bicarbonate, potassium carbonate, calcium bicarbonate, calcium carbonate, and combinations thereof; and wherein the effervescent system has a molar ratio of (i) the acid component to (ii) the alkaline component of about 1:1 to about 1:4.

In another aspect, the present disclosure provides a powdered effervescent laxative composition, the powdered composition comprising: about 70 weight percent or more of polyethylene glycol having a weight average molecular weight of between about 3,000 and about 4,000 g/mol; an effervescent system including (i) an acid component selected from the group consisting of monosodium citrate, disodium citrate, trisodium citrate, maleic acid, fumaric acid and combinations thereof and (ii) an alkaline component selected from the group consisting of sodium bicarbonate, sodium carbonate, potassium bicarbonate, potassium carbonate, calcium bicarbonate, calcium carbonate, and combinations thereof; and wherein the effervescent system has a molar ratio of (i) the acid component to (ii) the alkaline component of about 1:1 to about 1:4 wherein the product comprising less than 30 ppm of formaldehyde after storage in a closed container at about 40° C. and 75% relative humidity for about 8 weeks.

In another aspect, the present disclosure provides a powdered effervescent laxative composition, the powdered composition comprising: PEG 3350 and an effervescent system including monosodium citrate and sodium bicarbonate; wherein the composition comprises about 75 to about 90 weight percent of the PEG3350, about 4 to about 12 weight percent of the monosodium citrate, and about 4 to about 10 weight percent of the sodium bicarbonate; and wherein the effervescent system has a molar ratio of monosodium citrate to sodium bicarbonate of from about 1:1.5 to about 1:3.

In another aspect, the present disclosure provides a method of treating constipation in a subject in need thereof, comprising administering to the subject a composition, or a dose of a composition, as disclosed herein.

In another aspect, the present disclosure provides a method of softening stool in a subject in need thereof, comprising administering to the subject a composition, or a dose of a composition, as disclosed herein.

In another aspect, the present disclosure provides a method of increasing the frequency of bowel movements in a subject in need thereof, comprising administering to the subject a composition, or a dose of a composition, as disclosed herein.

In another aspect, the present disclosure provides a method of relieving occasional constipation in a subject in need thereof, comprising administering to the subject a composition, or a dose of a composition, as disclosed herein.

### DETAILED DESCRIPTION

As used herein, the following definitions shall apply unless otherwise indicated.

As used herein, “PEG” stands for “polyethylene glycol”, which is a polymeric ethylene oxide molecule having the general structure

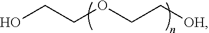

wherein “n” is an integer that is greater than 0. PEG nomenclature can include a number, such as “PEG 3350”, wherein the number indicates the weight-average and/or approximate molecular weight of the polymer.

As used herein, the term “laxative” refers to a drug or medicine that has the effect of stimulating or facilitating evacuation of the bowels in a subject.

As used herein the term “about” when preceding a numerical value, indicates that the numerical value can be ±5% of the total value indicated; for example, when a measured quantity disclosed herein is “about 100%”, the actual quantity could be from 95% to 105%. Unless specified otherwise, when the term “about” precedes a list of more than one numerical value, the term “about” is meant to be applied to all numerical values in the list.

As used herein, the term “all-inclusive” means that two or more, or all, components of a powdered effervescent laxative composition are comingled or mixed before packaging/storage in a ready-to-use package or dosage form.

Described herein is a powdered effervescent laxative composition. The composition includes about 70 weight percent or more of polyethylene glycol having a weight average molecular weight of between about 3,000 and about 4,000 g/mol. The composition also includes an effervescent system including (i) an acid component selected from the group consisting of monosodium citrate, disodium citrate, trisodium citrate, maleic acid, fumaric acid, adipic acid, and combinations thereof and (ii) an alkaline component selected from the group consisting of sodium bicarbonate, sodium carbonate, potassium bicarbonate, potassium carbonate, calcium bicarbonate, calcium carbonate, and combinations thereof. The effervescent system has a molar ratio of (i) the acid component to (ii) the alkaline component of from about 1:1 to about 1:4.

It is desirable to have a dry PEG composition comprising an effervescent agent pre-mixed together, i.e., in an all-inclusive, ready-to-use composition to provide simplicity for a user. The composition can be dissolved in water to be consumed quickly as a laxative treatment without additional pre-mixing steps. While ready-to-use effervescent compositions comprising PEG 3350 are available in single dose packets, it is difficult to provide a shelf-stable effervescent PEG composition, particularly that is stable in a multi-dose container.

In some embodiments, the effervescent system is all-inclusive.

In some embodiments, the effervescent system is ready-to-use.

In some embodiments, the composition remains shelf-stable in multi-dose containers.

In some embodiments, the powdered effervescent laxative includes polyethylene glycol (PEG) having a weight average molecular weight of between about 3015 g/mol to about 3685 g/mol.

In some embodiments, the PEG has a weight-average molecular weight about 3350 g/mol, which is known as PEG 3350.

In some embodiments, the weight/weight percentage of polyethylene glycol in composition is about 70% or more, or from about 70% to about 99%, or from about 80% to about 99%, or from about 80% to about 90%, or from about 80% to about 85%, or from about 82% to about 85%, or from about 82% to about 87%, or from about 82% to about 84%. In some embodiments, the composition comprises about 83.46% polyethylene glycol (e.g., PEG 3350).

In some embodiments, the acid component is monosodium citrate, disodium citrate, trisodium citrate, maleic acid, fumaric acid, and/or adipic acid.

In some embodiments, the acid component is monosodium citrate.

In some embodiments, the acid component is fumaric acid.

In some embodiments, the acid component is maleic acid.

In some embodiments, the acid component is disodium citrate.

In some embodiments, the acid component is trisodium citrate.

In some embodiments, the alkaline component is selected from the group consisting of sodium bicarbonate, sodium carbonate, potassium bicarbonate, potassium carbonate, calcium bicarbonate, calcium carbonate, and combinations thereof.

In some embodiments, the alkaline component is selected from the group of sodium bicarbonate, potassium bicarbonate, and calcium bicarbonate.

In some embodiments, the alkaline component is selected from the group of sodium carbonate, potassium carbonate, and calcium carbonate.

In some embodiments, the alkaline component is sodium bicarbonate.

In some embodiments, the alkaline component is potassium bicarbonate.

In some embodiments, the alkaline component is calcium bicarbonate.

In some embodiments, the alkaline component is sodium carbonate.

In some embodiments, the alkaline component is potassium carbonate.

In some embodiments, the alkaline component is calcium carbonate.

In some embodiments, the alkaline component is a surface-modified sodium bicarbonate that is surface-modified with sodium carbonate, for example, the substance Effer-Soda®, which is a highly stable, surface modified sodium bicarbonate powder, available from SPI Pharma.

In some embodiments, the acid component is monosodium citrate and the alkaline component is sodium bicarbonate.

In some embodiments, the molar ratio of (i) the acid component to (ii) the alkaline component is from about 1:1 to about 1:4, or from about 1:1 to about 1:3, or from about 1:1 to about 1:2. In some embodiments, the molar ratio of (i) monosodium citrate to (ii) sodium bicarbonate is from about 1:1 to about 1:4, or from about 1:1 to about 1:3, or from about 1:1 to about 1:2.

In some embodiments, the molar ratio of (i) the acid component to (ii) the alkaline component is from about 1:1.5 to about 1:3. In some embodiments, the molar ratio of (i) the acid component to (ii) the alkaline component is from about 1:1.7 to about 1:3. In some embodiments, the molar ratio of (i) the acid component to (ii) the alkaline component is from about 1:1.9 to about 1:2.5. In some embodiments, the molar ratio of (i) the acid component to (ii) the alkaline component is from about 1:1.9 to about 1:2.1. In some embodiments, the molar ratio of (i) the acid component to (ii) the alkaline component is about 1:2.

In some embodiments, the molar ratio of (i) monosodium citrate to (ii) sodium bicarbonate is from about 1:1.5 to about 1:3. In some embodiments, the molar ratio of (i) monosodium citrate to (ii) sodium bicarbonate is from about 1:1.7 to about 1:3. In some embodiments, the molar ratio of (i) monosodium citrate to (ii) sodium bicarbonate is from about 1:1.9 to about 1:2.5. In some embodiments, the molar ratio of (i) monosodium citrate to (ii) sodium bicarbonate is from about 1:1.9 to about 1:2.1. In some embodiments, the molar ratio of (i) monosodium citrate to (ii) sodium bicarbonate is about 1:2.

In some embodiments, the acid component comprises 50% or greater, 60% or greater, 70% or greater, 80% or greater, or 90% or greater of monosodium citrate.

In some embodiments, the acid component consists essentially of monosodium citrate.

In some embodiments, the term “consisting essentially of” may indicate that the acid component may comprise other acids, e.g., trace amounts of acids that do not affect the basic and novel characteristics of the effervescent system. For example, citric acid, formic acid, acetic acid, and/or oxalic acid may be present in trace to minor amounts e.g., less than 0.5% by weight of the composition.

In some embodiments, the effervescent system may comprise a second acid component, e.g., citric acid, tartaric acid, malic acid, or combinations thereof. The second acid component may be a minor component of the system. For example, the amount of citric acid may be less than 5% of the total weight of the composition, or about 4.5% or less, or about 4% or less, or about 3.5% or less, or about 3% or less, or about 2.5% or less, or about 2% or less, or about 1.5% or less, or about 1% or less, or about 0.5% or less. Also, for example, the amount of tartaric or malic acid may be less than 0.5% of the total weight of the composition, or about 0.25% or less, or about 0.1% or less.

In some embodiments, the composition is stable to heat and humidity over an extended time. When stored in a closed container at about 40° C. and 75% relative humidity for about 8 weeks, the composition comprises 30 ppm or less of formaldehyde (CH₂═O), or less than 20 ppm or formaldehyde, or less than 10 ppm of formaldehyde.

In some embodiments, when stored in a closed container at about 40° C. and 75% relative humidity for about 8 weeks, the concentration of total aldehydes formed, e.g., formaldehyde and acetaldehyde, is less than 200 ppm, or less than 150 ppm, or less than 125 ppm.

In some embodiments, the composition may have a weight:weight ratio of the polyethylene glycol to the acid component of from about 5:1 to about 20:1, or from about 8:1 to about 15:1, or from about 9:1 to about 11:1. In some embodiments, the composition may have a weight:weight ratio of the polyethylene glycol to monosodium citrate of from about 5:1 to about 20:1, or from about 8:1 to about 15:1, or from about 9:1 to about 11:1.

In some embodiments, the composition has a weight ratio of the polyethylene glycol to the acid component (i) of about 10:1. In some embodiments, the composition has a weight ratio of the polyethylene glycol to monosodium citrate of about 10:1.

In some embodiments, the composition has a weight ratio of the polyethylene glycol to the acid component (i) of about 9:1. In some embodiments, the composition has a weight ratio of the polyethylene glycol to monosodium citrate of about 9:1.

In some embodiments, the composition includes about 75 to about 90 weight percent of the polyethylene glycol, about 4 to about 12 weight percent of the (i) acid component, and about 4 to about 10 weight percent of the (ii) alkaline component.

In some embodiments, the composition includes 75 to about 90 weight percent of the polyethylene glycol, about 4 to about 12 weight percent of monosodium citrate, and about 4 to about 10 weight percent sodium bicarbonate.

In some embodiments, the composition includes about 4 to about 20 weight percent of monosodium citrate. In some embodiments, the composition includes about 4 to about 20 weight percent sodium bicarbonate. In some embodiments, the composition includes about 10 to about 20 weight percent of monosodium citrate. In some embodiments, the composition includes about 10 to about 20 weight percent sodium bicarbonate. In some embodiments, the composition includes about 7 to about 15 weight percent of monosodium citrate. In some embodiments, the composition includes about 4 to about 15 weight percent of sodium bicarbonate.

In some embodiments, the composition includes about 7 to about 12 weight percent of monosodium citrate. In some embodiments, the composition includes about 5 to about 10 weight percent sodium bicarbonate. In some embodiments, the composition includes about 7 to about 12 weight percent of monosodium citrate and about 5 to about 10 weight percent sodium bicarbonate. In some embodiments, the composition includes about 7 to about 12 weight percent of monosodium citrate and about 4 to about 10 weight percent sodium bicarbonate. In some embodiments, the composition includes about 8 to about 11 weight percent of monosodium citrate and about 6 to about 9 weight percent sodium bicarbonate. In some embodiments, the composition includes about 7 to about 11 weight percent of monosodium citrate and about 5 to about 9 weight percent sodium bicarbonate. In some embodiments, the composition includes about 7 to about 10 weight percent of monosodium citrate and about 5 to about 8 weight percent sodium bicarbonate. In some embodiments, the composition includes about 9 to about 10 weight percent of monosodium citrate (e.g., 9.27 weight percent) and about 7 to about 8 weight percent sodium bicarbonate (e.g., 7.27 weight percent). In some embodiments, the composition includes about 8 to about 10 weight percent of monosodium citrate (e.g., 8.18 weight percent) and about 6 to about 8 weight percent sodium bicarbonate (e.g., 6.43 weight percent). In some embodiments, the composition includes about 8 to about 9.5 weight percent of monosodium citrate and about 6 to about 7.5 weight percent sodium bicarbonate.

In some embodiments, the composition may comprise a weight ratio of the polyethylene glycol to the acid component (i) of from about 8.8:1 to about 12:1 and a molar ratio of (i) the acid component to (ii) the alkaline component of from about 1:1.9 to about 1:2.5.

In some embodiments, the composition may comprise a weight ratio of the polyethylene glycol to monosodium citrate (i) of from about 8.8:1 to about 12:1 and a molar ratio of (i) monosodium citrate to (ii) sodium bicarbonate of from about 1:1.9 to about 1:2.5.

In some embodiments, the composition is shelf-stable for at least 4 weeks, or at least 8 weeks, or at least 12 weeks, or at least 6 months, at 40° C. and 75% relative humidity.

In some embodiments, the composition is shelf-stable for at least 30 days or at least 90 days when stored uncapped or when stored with a cap with a broken seal or no seal at 25° C. and 60% relative humidity.

In some embodiments, the composition may comprise a flavoring agent and/or a sweetener and/or a colorant.

In some embodiments, the flavoring agent imparts a flavor selected from but not limited to lime, orange, cherry, grape, strawberry, raspberry, watermelon, blueberry, vanilla, hazelnut, chocolate, coffee, peppermint, caramel, fruit punch, and lemonade to the composition.

In some embodiments, the flavoring agent imparts a lime and/or margarita flavor to the composition. In still a further embodiment, the flavoring agent is natural lime which is an arabic gum-based flavor. In some embodiments, the composition is flavored with a small amount of an acid, such as citric acid, ascorbic acid, tannic acid, lactic acid, malic acid, or tartaric acid.

In some embodiments, the composition comprises from 0.01 weight percent to 10 weight percent of a flavoring agent. In a further embodiment, the composition comprises from 0.1 weight percent to 2.0 weight percent of a flavoring agent. In still a further embodiment, the composition comprises from 0.2 weight percent to 1.0 weight percent of a flavoring agent. In yet a further embodiment, the composition comprises about 0.49 weight percent of a flavoring agent.

In some embodiments, the composition comprises a sweetener.

In some embodiments, the sweetener is selected from but not limited to glucose, sucrose, maltose, mannose, dextrose, fructose, lactose, trehalose, maltitol, lactitol, xylitol, sorbitol, mannitol, tagatose, glycerin, erythritol, isomalt, maltose, sucralose, aspartame, neotame, alitame, neohesperidin dihydrochalcone, cyclamate, thaumatin, acesulfame potassium, saccharin, saccharin sodium or a combination thereof. In still a further embodiment, the sweetener is sucralose.

In some embodiments, the composition comprises from 0 weight percent to 10 weight percent of a sweetener. In a further embodiment, the composition comprises from 0.01 weight percent to 1.0 weight percent of a sweetener. In still a further embodiment, the composition comprises from 0.01 weight percent to 0.5 weight percent of a sweetener. In yet a further embodiment, the composition comprises about 0.19 weight percent of a sweetener.

Also provided herein is a packaged and shelf-stable powdered effervescent laxative composition. The packaged composition includes a sealed container and a powdered effervescent laxative composition contained in the sealed container. The powdered effervescent laxative composition includes about 70 weight percent or more of polyethylene glycol having a weight average molecular weight of from 3000 to 4000 g/mol. The composition also includes and an effervescent system including (i) an acid component selected from the group consisting of monosodium citrate, disodium citrate, trisodium citrate, maleic acid, fumaric acid, adipic acid, and combinations thereof and (ii) an alkaline component selected from the group consisting of sodium bicarbonate, sodium carbonate, potassium bicarbonate, potassium carbonate, calcium bicarbonate, calcium carbonate, and combinations thereof. The effervescent system has a molar ratio of (i) the acid component to (ii) the alkaline component of about 1:1 to about 1:4.

In some embodiments, the sealed container is a multi-dose or single-dose container.

In some embodiments, the sealed container is a bottle, a sachet, a pouch, a vial, and a blister pack.

In some embodiments, the sealed container is a bottle which has a cap and a seal.

In some embodiments, the bottle is formed of polyethylene (e.g., high density polyethylene), polypropylene, ethylene vinyl alcohol (EVOH), nylon, polyester, polyethylene terephthalate, glass, or combinations thereof.

In some embodiments, when the container is a pouch, the pouch may have one or more heat seals. In a further embodiment, the pouch may be formed of one or more layers selected from the group consisting of polyethylene, nylon, polyester, polypropylene, (meth)acrylate, (meth)acrylic acid, foil (e.g., aluminum foil), polyethylene terephthalate, metalized derivatives thereof, and combinations thereof.

In some embodiments, the container is a pouch having one or more heat seals.

In some embodiments, the container has 1, 2, 3, or 4 heat seals.

In some embodiments, the sealed container is a multi-dose container which contains multiple doses of the powdered effervescent laxative composition.

In some embodiments, the acid component (i) of the packaged composition consists essentially of monosodium citrate.

In some embodiments, the packaged composition has a weight ratio of the polyethylene glycol to the acid component (i) of about 8:1 to about 15:1.

In some embodiments, the packaged composition comprises about 75 to about 90 weight percent of the polyethylene glycol, about 4 to about 12 weight percent of the (i) acid component, and about 4 to about 10 weight percent of the (ii) alkaline component. In a further embodiment the weight ratio of the polyethylene glycol to the acid component (i) is about 8.8:1 to about 12:1 and the molar ratio of (i) the acid component to (ii) the alkaline component is about 1:1.2 to about 1:1.5.

In some embodiments, the packaged composition may remain shelf-stable for at least 4 weeks at 40° C. and 75% relative humidity.

In some embodiments, there is provided powdered effervescent laxative composition, the powdered composition comprising about 70 weight percent or more of polyethylene glycol having a weight average molecular weight of between about 3,000 and about 4,000 g/mol; and comprising an effervescent system including (i) an acid component selected from the group consisting of monosodium citrate, disodium citrate, trisodium citrate, maleic acid, fumaric acid and combinations thereof and (ii) an alkaline component selected from the group consisting of sodium bicarbonate, sodium carbonate, potassium bicarbonate, potassium carbonate, calcium bicarbonate, calcium carbonate, and combinations thereof; and wherein the effervescent system has a molar ratio of (i) the acid component to (ii) the alkaline component of about 1:1 to about 1:4, wherein the product comprises less than 30 ppm of formaldehyde after storage in a closed container at about 40° C. and 75% relative humidity for about 8 weeks.

In some embodiments of the powdered effervescent laxative composition, the molar ratio of (i) the acid component to (ii) the alkaline component is from about 1:1.5 to about 1:3. In a further embodiment, the molar ratio of (i) the acid component to (ii) the alkaline component is from about 1:1.7 to about 1:3. In yet another embodiment, the molar ratio of (i) the acid component to (ii) the alkaline component is from about 1:1.9 to about 1:2.5. In yet another further embodiment, the molar ratio of (i) the acid component to (ii) the alkaline component is from about 1:1.9 to about 1:2.1.

In some embodiments, the molar ratio of (i) the acid component to (ii) the alkaline component is about 1:2.

In some embodiments, the alkaline component is selected from the group consisting of sodium bicarbonate, potassium bicarbonate, and calcium bicarbonate.

In some embodiments, the alkaline component is sodium bicarbonate.

In some embodiments, the alkaline component is potassium bicarbonate.

In some embodiments, the alkaline component is calcium bicarbonate.

In some embodiments, the alkaline component is sodium carbonate.

In some embodiments, the alkaline component is potassium carbonate.

In some embodiments, the alkaline component is calcium carbonate.

In some embodiments, the alkaline component is a surface-modified sodium bicarbonate powder that is surface-modified with sodium carbonate, e.g., Effer-Soda®.

In another aspect, the present disclosure provides a powdered effervescent laxative composition, the powdered composition comprising: about 70 weight percent or more of PEG 3350; and includes an effervescent system including (i) monosodium citrate and (ii) sodium bicarbonate; wherein the effervescent system has a molar ratio of (i) monosodium citrate to (ii) sodium bicarbonate of from about 1:1.5 to about 1:3.

In another aspect, the present disclosure provides a packaged and shelf-stable powdered effervescent laxative composition in a multi-dose container, the packaged composition comprising: a sealed multi-dose container and a powdered effervescent laxative composition contained in the sealed multi-dose container; wherein the powdered effervescent laxative composition includes about 70 weight percent or more of PEG 3350; and includes an effervescent system including (i) monosodium citrate and (ii) sodium bicarbonate; wherein the effervescent system has a molar ratio of (i) monosodium citrate to (ii) sodium bicarbonate of about 1:1.5 to about 1:3.

In another aspect, the present disclosure provides a powdered effervescent laxative composition, the powdered composition comprising: PEG 3350 and an effervescent system including monosodium citrate and sodium bicarbonate; wherein the composition comprises about 75 to about 90 weight percent of the PEG3350, about 4 to about 12 weight percent of the monosodium citrate, and about 4 to about 10 weight percent of the sodium bicarbonate; and wherein the effervescent system has a molar ratio of monosodium citrate to sodium bicarbonate of from about 1:1.5 to about 1:3.

In some embodiments, the effervescent system has a molar ratio of monosodium citrate to sodium bicarbonate of about 1:2. In some embodiments, the composition comprises from about 8 to about 11 weight percent of monosodium citrate and from about 6 to about 9 weight percent sodium bicarbonate. In some embodiments, the composition comprises from about 7 to about 11 weight percent of monosodium citrate and from about 5 to about 8 weight percent sodium bicarbonate. In some embodiments, the composition comprises from about 7 to about 10 weight percent of monosodium citrate and from about 5 to about 8 weight percent sodium bicarbonate. In some embodiments, the composition comprises from about 9 to about 10 weight percent of monosodium citrate and from about 7 to about 8 weight percent sodium bicarbonate. In some embodiments, the composition comprises from about 8 to about 10 weight percent of monosodium citrate and from about 6 to about 8 weight percent sodium bicarbonate. In some embodiments, the composition comprises from about 8 to about 9.5 weight percent of monosodium citrate and from about 6 to about 7.5 weight percent sodium bicarbonate.

PEG, and PEG3350 in particular, is useful for treating constipation, softening stool, increasing frequency of bowel movements, and relieving occasional constipation. In one aspect, the present disclosure provides a method of treating constipation in a subject in need thereof, comprising administering to the subject a composition, or a dose of a composition, as disclosed herein. In another aspect, the present disclosure provides a method of softening stool in a subject in need thereof, comprising administering to the subject a composition, or a dose of a composition, as disclosed herein. In another aspect, the present disclosure provides a method of increasing the frequency of bowel movements in a subject in need thereof, comprising administering to the subject a composition, or a dose of a composition, as disclosed herein. In another aspect, the present disclosure provides a method of relieving occasional constipation in a subject in need thereof, comprising administering to the subject a composition, or a dose of a composition, as disclosed herein.

### EXAMPLES

### Analytical Procedures

The following procedures were used in the experiments to generate the data described in the Examples herein.

Description (Visual examination): The physical appearance of the sample was visually evaluated against the product specification for color and texture.

Container/Closure (Visual examination): The container seal was examined to confirm the seal is intact, the container is tightly sealed and there has been no chemical interaction with the product and the container/container seal. The seal was removed and the appearance of the sample in the container was evaluated to confirm no change to the product appearance. The sample was removed from the container and the empty container was examined to determine if there had been any chemical interaction between the product and the container. The sample outside of the container is visually evaluated as a final confirmation no change in appearance/color or texture has occurred.

### Acetic Acid and Formic Acid (by HPLC):

High Pressure Liquid Chromatography analyses were performed using a Waters Alliance 2690/5 Separation Module. The Alliance 2690/5 is an integrated solvent and sample management platform equipped with a gradient pump, separation module, a column heater/chiller and a two-channel 2489 ultraviolet/visible (UV/Vis) detector. The sample management system consists of five carousels that hold twenty-four 2 mL glass vials. The sample was injected into the flow path of the separation module via a standard 250-μL syringe. A Restek Alure Organic Acids 5 μm, 4.6×300 mm analytical column was installed. 0.7 mL of 85% H3PO4 (MW 97.994): 1000 mL of HPLC water (18.2 MW) was used to fill eluent reservoir A. Acetonitrile (41.053 MW) was used to fill eluent reservoir B. HPLC water was used to dilute the sample, standard solutions, and for the blank injections. 5% Acetonitrile: 95% HPLC water was used for the autosampler flush.
Wavelength: 210 nm
Run Time: 36 min
Flow rate: 1.000 mL/min
Injection Volume: 10 μL
Data Collection Rate: 10 points/sec
Column Temperature: 25° C.
Autosampler Temperature: ambient

Dispersibility (Visual): An evaluation of the ability of the product to disperse quickly and evenly in water was performed by adding a sample containing 17 g of PEG3350 into 118-237 mL of room temperature DI water and stirring. The resulting solution should be free of undispersed powder (conglomerates). The typical dissolution time is 3.5 minutes or less.

### Assay (by Size Exclusion Chromatography, SEC):

Size exclusion chromatography by optical refraction were performed using a Waters Alliance 2690/5 Separation Module. The Alliance 2690/5 is an integrated solvent and sample management platform equipped with a gradient pump, separation module, a column heater/chiller and a Waters 2414 Refractive Index Detector. A Ultrahydrogel DP 6 μm, 7.8 mm×300 mm column equipped with a Ultrahydrogel DP 6 μm, 6 mm×40 mm guard column was installed. The sample was introduced into the separation module through the sample management system which consist of five carousels that hold twenty-four 2 mL glass vials. The sample was injected into the flow path of the separation module via a standard 250-μL syringe. 0.005% Sodium Azide in HPLC water solution was used to fill eluent reservoir A. 0.05% Sodium Azide in HPLC water solution was used to fill eluent reservoir B. Acetonitrile (41.053 MW) and HPLC water (18.2 MW) were used to prepare the sample and standards.
Run Time: 18 min
Flow rate: 0.8 mL/min
Injection Volume: 20 μL
Data Collection Rate: 2 points/sec
Polarity: (+)
Column Temperature: 35° C.
Detector Temperature: 35° C.
Autosampler Temperature: ambient

### Limit of Formaldehyde and Acetaldehyde (By HPLC):

High Pressure Liquid Chromatography analyses were performed using a Waters Alliance 2690/5 Separation Module. The Alliance 2690/5 is an integrated solvent and sample management platform equipped with a gradient pump, separation module, a column heater/chiller and a two-channel 2489 ultraviolet/visible (UV/Vis) detector. A Zorbax Eclipse XDB C8, 3.5 μm 3.0 mm×150 mm was installed. The integrated sample management system consists of five carousels that hold twenty-four 2 mL glass vials. The sample was injected into the flow path of the separation module via a standard 250-μL syringe. HPLC water (18.2 MW) was used to fill reservoir A. Acetonitrile (41.053 MW) was used to fill reservoir B.

Formaldehyde and Acetaldehyde Standard solutions for the analysis were prepared by diluting Formaldehyde-DNPH Solution 100 μg/mL and Acetaldehyde-DNPH Solution 1000 μg/mL with Acetonitrile.

A 5 mg/mL 2,4-Dinitrophenylhydrazine (DNPH) in acidified (Hydrochloric Acid) Acetonitrile Solution was prepared. A sample weight equivalent to 500 mg of PEG 3350 was weighed into a 10 mL volumetric flask. The sample was dissolved in 1.0 mL of Acetonitrile and 2.0 mL of DNPH was pipetted into the flask. A DNPH Blank was similarity prepared by spiking 2.0 mL of DNPH into 1.0 mL of Acetonitrile. The preparations were allowed to react for 15 minutes and then diluted to volume with Acetonitrile. Aliquots of sample and blank solution were transferred to glass vials and quantified in the Alliance 2690/5. The aldehyde concentrations detected in the DNPH blank were subtracted from the aldehyde concentration in the sample and the results reported.
Wavelength: 360 nm
Run Time: 17 min
Flow rate: 0.65 mL/min (using gradient conditions)
Injection Volume: 5 μL
Data Collection Rate: 10 points/sec
Column Temperature: 30° C.
Autosampler Temperature: ambient

Water Content: The water content was determined by calculating the percentage of weight lost while drying over silica. The analytical balance was tared. A shallow weigh dish was placed onto the balance and 5 g of sample was weighed into the weigh dish. The weight of the dish and the sample was recorded. The weigh dish was placed into a desiccator containing silica gel and allowed to dry in the desiccator for twenty-four hours. The weigh dish containing the dried sample was then weighed on an analytical balance and recorded. The difference between the dried and undried sample was calculated. The water content was calculated by dividing the difference between the undried and dried samples by the gross weight of the undried sample.

ID-A (by Attenuated Total Reflection, ATR): Attenuated Total Reflection (ATR) Spectroscopy analysis was performed on a Thermo Scientific Nicolet iS50 spectrophotometer equipped with a Nicolet is50 FTIR ATR using OMNIC FTIR software. The FTIR daily verification was performed using the built-in ValPro Qualification Software. A few milligrams of sample were placed on the ATR crystal window, the anvil arm was locked and the sample was compressed onto the window. The sample was scanned over the range of 3800 cm-1 to 650 cm-1. Transmission spectra were collected with 32 sample scans and 4 cm-1 resolution at an optical velocity of 0.4747 cm/sec. A valid, non-expired USP Reference Standard was similarly scanned to generate a reference spectrum. The sample spectrum was compared against the reference spectrum.

ID-B (Retention time by SEC): The Polyethylene Glycol assay HPLC analysis was used to assess the retention time of the sample relative to the retention time of the reference standard. The average retention time of the active pharmaceutical ingredient (minutes) of the assay standard (or system suitability), was calculated from the standard injection prior to the sample injection and the standard (or system suitability) injection after the sample injection. The average retention time of the standard was then subtracted from the average retention time of the active pharmaceutical ingredient for the sample to determine the difference. The difference in the sample and standard retention time was then divided by the average retention time of the standard to calculate the % Difference between the standard and sample retention times. A % Difference of not more than 2.0% was considered passing.

### Example 1: Incompatibility of PEG with Citric Acid

### Procedure:

Mixes were prepared in a blender, and approximately half the quantity of Polyethylene Glycol 3350 (PEG) API, flavor, sweetener, acid (Citric Acid), base (Sodium Bicarbonate or Effer-Soda®) and the remaining half quantity of Polyethylene Glycol 3350 (PEG) API were added to the V-shell blender and mixed. Various sized screens were used to delump the acid and base components. Bulk mixes were filled into multi-dose bottles, pouches, or vials.

### Batches:

Citric Acid Batch 1:

| | | Weight |
|---|---|---|
| Ingredient | mg/dose | percent |
| PEG 3350 | 17000 | 89.2 |
| Orange Flavor | 100 | 0.5 |
| Sucralose | 14 | 0.1 |
| Citric Acid | 839 | 4.4 |
| Sodium Bicarbonate | 1101 | 5.8 |

Citric Acid Batch 2:

| | | Weight |
|---|---|---|
| Ingredient | mg/dose | percent |
| PEG 3350 | 17000 | 88.4 |
| Orange Flavor | 100 | 0.5 |
| Sucralose | 14 | 0.1 |
| Citric Acid | 839 | 4.4 |
| Effer-Soda ® | 1273 | 6.6 |

### Testing and Results:

Citric Acid Batches 1 and 2 were tested for stability under accelerated and room temperature conditions under several different packaging configurations, namely, 7- and 45-daily dose bottles, pouches, and vials with DesiCaps®. The testing data is shown in the table below.

| | | | | Description | | | | |
|---|---|---|---|---|---|---|---|---|
| | | | | White or almost | | | | |
| | | | | white solid with | Water | | | Total |
| | | | | waxy or | Content | | | Aldehydes |
| | | | Time | paraffin-like | NMT | pH | Formaldehyde | NMT 200 |
| Batch | Package | Condition | (Months) | appearance | 1.0% | 4.5-7.5 | NMT 30 ppm | ppm |
| Citric | Mix | Initial | 0 | Orange, Waxy | 1.2% | 6.3 | — | — |
| Acid | | | | Appearance | | | | |
| Batch | 7-daily | 25C/ | 3 | — | — | — | 8 | 68 |
| 1 | dose | 60% RH | | | | | | |
| | bottle | | | | | | | |
| | 45-daily | | | — | — | — | 8 | 78 |
| | dose | | | | | | | |
| | bottle | | | | | | | |
| | Pouch | 40C/ | 1 | Fail: Atypical | 1.6% | 5.5 | — | — |
| | | 75% RH | | (lumped/ | | | | |
| | | | | conglomerated) | | | | |
| | | | 2 | Fail: Pouches | — | — | — | — |
| | | | | expanded, | | | | |
| | | | | samples | | | | |
| | | | | discarded, | | | | |
| | | | | study ended | | | | |
| | Vial | 40C/ | 1 | Fail: Atypical | 0.9% | 6 | — | — |
| | with | 75% RH | | (lumped/ | | | | |
| | Desi-cap | | | conglomerated) | | | | |
| | | | 2 | — | — | — | 31 | 70 |
| | | | 3+ | Fail: Atypical | 0.4% | 6.3 | 106 | 141 |
| Citric | Mix | Initial | 0 | Orange, Waxy | 1.0% | 6.3% | — | — |
| Acid | | | | Appearance | | | | |
| Batch | 7-daily | 40C/ | 1 | Fail: Atypical | 0.9% | 8.5 | — | — |
| 2 | dose | 75% RH | | (lumped/ | | | | |
| | bottle | | | conglomerated) | | | | |
| | | | 3+ | Fail: Atypical | 1.6% | 7.1 | 72 | 125 |
| | 14-daily | 40C/ | 1 | Fail: Atypical | 1.3% | 6.4 | — | — |
| | dose | 75% RH | | (lumped/ | | | | |
| | bottle | | | conglomerated) | | | | |
| | | | 3+ | Fail: Atypical | 1.6% | 6.9 | 64 | 112 |
| | | 25C/ | 3 | — | — | — | 10 | 81 |
| | | 60% RH | | | | | | |
| | 30-daily | 40C/ | 1 | Fail: Atypical | 1.4% | 6.9 | — | — |
| | dose | 75% RH | | (lumped/ | | | | |
| | bottle | | | conglomerated) | | | | |
| | | | 3+ | Fail: Atypical | 1.0% | 6.6 | 53 | 102 |
| | 45-daily | 40C/ | 1 | Fail: Atypical | 1.2% | 8.0 | — | — |
| | dose | 75% RH | | (lumped/ | | | | |
| | bottle | | | conglomerated) | | | | |
| | | | 3+ | Fail: Atypical | 1.1% | 7.6 | 54 | 102 |
| | | 25C/ | 3 | — | — | — | 49 | 112 |
| | | 60% RH | | | | | | |
| | Pouch | 40C/ | 1 | Fail: Atypical | 1.7% | 4.5 | — | — |
| | | 75% RH | | (lumped/ | | | | |
| | | | | conglomerated) | | | | |
| | | | 2 | — | — | — | 31, 85 | 79, 104 |
| | | | 3+ | Fail: Atypical | 0.7% | 7.1 | 45 | 87 |
| | Vial | 40C/ | 1 | Fail: Atypical | 1.0% | 6.3 | — | — |
| | with | 75% RH | | (lumped/ | | | | |
| | Desi-cap | | | conglomerated) | | | | |
| | | | 2 | — | — | — | 81 | 102 |
| | | | 3+ | Fail: Atypical | 0.7% | 7.1 | 130 | 131 |

Citric Acid Batch 1 in the pouch failed testing for visual description and water content after 1 month at 40 C/75% RH. The pouch sample also showed significant expansion. Description for Citric Acid Batch 1 in vial with DesiCap® also failed after 1 month at 40 C/75% RH. Formaldehyde and total acetaldehyde levels were passing at 2 months at 40 C/75% RH in the vial with DesiCap® but formaldehyde failed after 3 months at 40 C/75% RH. Citric Acid Batch 2 failed the formaldehyde specification in all packaging configurations tested after 3 months at 40 C/75% RH. Overall, this study indicated that changing the packaging type from multi-dose bottles (40 C/75% RH data not shown for another batch of the same composition of batch 1—failure observed after 1 month on storage) to pouches or to vials with DesiCaps® did not achieve the desired description or impurity results, with the Citric acid and Sodium Bicarbonate or Effer-Soda® composition.

### Example 2: Excipient Compatibility Study (ECS)

### Procedure:

Other acidic components were tested in place of citric acid to investigate whether they had improved compatibility with PEG. Binary mixtures of the components with PEG were prepared and tested for stability under accelerated conditions. Each study was conducted with a control sample (Orange PEG only) that was stressed side by side with the binary mixtures. The ECS samples were prepared by first passing the excipients through a 20-mesh screen, weighing the ingredients to the specific amount and transferring the ingredients into an HDPE bottle and manually mixing the bottle for 5 minutes. Then aliquots were prepared from the bulk blends by weighing specified amounts into clear glass vials which were tightly closed.

### Testing and Results:

All closed vials were loaded into the 40 C/75% RH stability chamber and stressed for the specified amount of time, at which point samples were removed from the chamber and submitted for analysis. Fumaric acid and monosodium citrate showed little to no formaldehyde impurity growth. The citric acid sample showed very high (OOS) formaldehyde levels starting at two weeks and total aldehydes OOS at eight weeks. Results are shown in the table below.

| | | | Description | | |
|---|---|---|---|---|---|
| | | | White or almost | | Total |
| | | | white solid with | | Aldehydes |
| | | | a waxy or | Formaldehyde | (ppm) |
| | Storage | | paraffin-like | (ppm) | NMT 200 |
| Time Point | Condition | Sample | appearance | NMT 30 ppm | ppm |
| Initial | 25C/60% RH | Orange PEG | Pass: White, | 7 | 86 |
| | | Only Control | Waxy Substance | | |
| 2 weeks | 40C/75% RH | | Pass: White, | 4 | 78 |
| | | | Waxy Substance | | |
| 8 weeks | 40C/75% RH | | Fail: Atypical, | 3 | 69 |
| | | | slight clumping | | |
| Initial | 25C/60% RH | 10:1 Orange | Pass: White, | 7 | 84 |
| | | PEG:Citric | Waxy Substance | | |
| 2 weeks | 40C/75% RH | Acid | Fail: Atypical | 153 | 191 |
| | | | white with slight | | |
| | | | clumping | | |
| 8 weeks | 40C/75% RH | | Fail: Atypical, | 262 | 274 |
| | | | agglomeration | | |
| | | | to waxy paste | | |
| Initial | 25C/60% RH | Orange PEG | Pass: White | 8 | 91 |
| | | Only Control | solid with waxy | | |
| | | | appearance | | |
| 2 weeks | 40C/75% RH | | Fail: Atypical | 7 | 86 |
| | | | waxy, white | | |
| | | | appearance | | |
| 4 weeks | 40C/75% RH | | Pass: White | 8 | 88 |
| | | | solid with waxy | | |
| | | | appearance | | |
| Initial | 25C/60% RH | 10:1 Orange | Pass: White | 8 | 89 |
| | | PEG:Monosodium | solid with waxy | | |
| | | Citrate D | appearance | | |
| 2 weeks | 40C/75% RH | | Fail: Atypical | 8 | 87 |
| | | | waxy, white | | |
| | | | appearance | | |
| 4 weeks | 40C/75% RH | | Pass: White | 9 | 78 |
| | | | solid with waxy | | |
| | | | appearance | | |
| Initial | 25C/60% RH | Orange PEG | Pass: White | 8 | 92 |
| | | Only Control | solid with waxy | | |
| | | | appearance | | |
| 2 weeks | 40C/75% RH | | Pass: White | 9 | 90 |
| | | | solid with waxy | | |
| | | | appearance | | |
| 4 weeks | 40C/75% RH | | Pass: White | 7 | 83 |
| | | | solid with waxy | | |
| | | | appearance | | |
| Initial | 25C/60% RH | 10:1 Orange | Pass: White | 8 | 89 |
| | | PEG:Fumaric | solid with waxy | | |
| | | Acid | appearance | | |
| 2 weeks | 40C/75% RH | | Pass: White | 14 | 44 |
| | | | solid with waxy | | |
| | | | appearance | | |
| 4 weeks | 40C/75% RH | | Pass: White, | 13 | 50 |
| | | | waxy substance | | |

### Example 3: Monosodium Citrate as the Acidic Component of Effervescent Couple

### Procedure:

A pouch batch was prepared individually with Monosodium Citrate D (no final mix). Monosodium Citrate D and sodium bicarbonate were each screened through a #30 mesh. The batch was prepared by making small scale (75 g) mixtures of the acidic component and the basic component, with bag mixing, then this acid/base mixture was added to pouches with Orange PEG powder. The ingredients and amounts of the batch were as follows:

| Ingredient | mg/dose | Weight percent |
|---|---|---|
| Orange PEG Powder | 17114 | 83.46 |
| Monosodium Citrate D | 1900 | 9.27 |
| Sodium Bicarbonate | 1491 | 7.27 |

### Testing and Results:

The pouches were heat sealed and placed in 40 C/75% RH stability chambers.

| | | | Dispersibility | | | | |
|---|---|---|---|---|---|---|---|
| | | Description | Material | | | | |
| | | White or | completely | | | | |
| | | almost white | dissolves with | | | | |
| | | solid with | mixing. | Water | | | Total |
| | | waxy or | Solution may | Content | | | Aldehydes |
| | Time | paraffin-like | be slightly | NMT | pH | Formaldehyde | NMT 200 |
| Condition | (weeks) | appearance | hazy | 1.0% | 4.5-7.5 | NMT 30 ppm | ppm |
| Initial | 0 | White solid | — | 0.2% | 6.2 | 10 | 107 |
| | | with waxy | | | | | |
| | | appearance | | | | | |
| 40C/ | 2 | White solid | — | 0.2% | 6.3 | 7 | 73 |
| 75% RH | | with waxy | | | | | |
| | | appearance | | | | | |
| | 4 | White solid | Pass | 0.4% | 6.4 | 11 | 109 |
| | | with waxy | | | | | |
| | | appearance | | | | | |
| | 8 | White solid | Pass | 0.7% | 6.1 | 8 | 78 |
| | | with waxy | | | | | |
| | | appearance | | | | | |

Formulation composition with Monosodium Citrate D (1900 mg/dose) and Sodium Bicarbonate (1491 mg/dose) was stable after 8 weeks at 40 C/75% RH. All properties tested (description, dispersibility, water content, pH, formaldehyde and total aldehydes) were within specification acceptance criteria. A parallel formulation composition tested with Effer-Soda® in place of sodium bicarbonate was also stable after 4 weeks at 40 C/75% RH.

### Example 4: In Process Use Study

An in-process use study was designed to mimic the way a consumer uses the product, with one dose taken every day. The results showed that even under the harshest condition critical parameters, water and impurities remained well within proposed specification. Testing was performed at 3 conditions: (1) 25 C/60% RH open (no cap) “uncapped”, (2) 25 C/60% RH re-capped but seal broken “capped”, and (3) at ambient conditions on the bench “ambient”. A batch of the composition was prepared as shown in the table below and multiple doses of the composition were placed in containers to be tested.

| Ingredient | Weight percent |
|---|---|
| PEG 3350 | 82.80% |
| Sucralose | 0.20% |
| Monosodium Citrate | 9.25% |
| Sodium Bicarbonate | 7.26% |
| Citrus Flavor | 0.49% |

The study included a 30-day study where one dose was removed every day and images were taken at 30 days. The study also included a 90-day study where two samples were removed per week. Description and dispersibility were recorded at every pour, and the effervescence noted as well. The sample remained readily flowable with no agglomeration and had similar levels of effervescence throughout the study. The data from the 30-day and 90-day studies are shown in the tables below.

### 30-Day Study:

| Time | | | Container/ | | Water | Formic and |
|---|---|---|---|---|---|---|
| Point | Condition | Description | Closure | Assay | Content | Acetic Acid |
| T0 | N/A | White solid with | N/A | 100.2 | 0.30% | NMT 5000 ppm |
| | | waxy appearance | | | | |
| 15-Day | Ambient | White solid with | Pass | 101.8 | 0.10% | NMT 5000 ppm |
| | | waxy appearance | | | | |
| 15-Day | Capped | White solid with | Pass | 102.4 | 0.10% | NMT 5000 ppm |
| | | waxy appearance | | | | |
| 15-Day | Uncapped | White solid with | Pass | 101.6 | 0.40% | NMT 5000 ppm |
| | | waxy appearance | | | | |
| 30-Day | Ambient | White solid with | Pass | 99.6 | 0.30% | NMT 5000 ppm |
| | | waxy appearance | | | | |
| 30-Day | Capped | White solid with | Pass | 99.9 | 0.40% | NMT 5000 ppm |
| | | waxy appearance | | | | |
| 30-Day | Uncapped | White solid with | Pass | 102.5 | 0.60% | NMT 5000 ppm |
| | | waxy appearance | | | | |

| | | | | | Total |
|---|---|---|---|---|---|
| Time | | | Formaldehyde | Acetaldehyde | Impurities |
| Point | Condition | Dispersibility | (ppm) | (ppm) | (ppm) |
| T0 | N/A | Pass | 18 | 84 | 102 |
| 15-Day | Ambient | Pass | 14 | 78 | 92 |
| 15-Day | Capped | Pass | 14 | 78 | 93 |
| 15-Day | Uncapped | Pass | 10 | 79 | 89 |
| 30-Day | Ambient | Pass | 13 | 75 | 88 |
| 30-Day | Capped | Pass | 13 | 76 | 89 |
| 30-Day | Uncapped | Pass | 6 | 78 | 84 |

### 90-Day Study:

| | | | Container/ | | Water | Formic and |
|---|---|---|---|---|---|---|
| Time Point | Condition | Description | Closure | Assay | Content | Acetic Acid |
| 30-Day | Ambient | White solid with | Pass | 101.6 | 0.20% | NMT 5000 ppm |
| | | waxy appearance | | | | |
| 30-Day | Capped | White solid with | Pass | 101.7 | 0.20% | NMT 5000 ppm |
| | | waxy appearance | | | | |
| 30-Day | Uncapped | White solid with | Pass | 103.1 | 0.60% | NMT 5000 ppm |
| | | waxy appearance | | | | |
| 60-Day | Ambient | White, waxy solid | Pass | 99.2 | 0.30% | NMT 5000 ppm |
| 60-Day | Capped | White, waxy solid | Pass | 102.1 | 0.60% | NMT 5000 ppm |
| 60-Day | Uncapped | White, waxy solid | Pass | 99.6 | 0.30% | NMT 5000 ppm |
| 90-Day | Ambient | White, waxy solid | Pass | 98.5 | 0.30% | NMT 5000 ppm |
| 90-Day | Capped | White, waxy solid | Pass | 98.7 | 0.40% | NMT 5000 ppm |
| 90-Day | Uncapped | White, waxy solid | Pass | 102.2 | 0.70% | NMT 5000 ppm |

| | | | Formaldehyde | Acetaldehyde | Total Impurities |
|---|---|---|---|---|---|
| Time Point | Condition | Dispersibility | (ppm) | (ppm) | (ppm) |
| 30-Day | Ambient | Pass | 13 | 75 | 88 |
| 30-Day | Capped | Pass | 13 | 74 | 87 |
| 30-Day | Uncapped | Pass | 7 | 75 | 82 |
| 60-Day | Ambient | Pass | 17 | 83 | 100 |
| 60-Day | Capped | Pass | 7 | 81 | 88 |
| 60-Day | Uncapped | Pass | 17 | 85 | 102 |
| 90-Day | Ambient | Pass | 17 | 84 | 101 |
| 90-Day | Capped | Pass | 17 | 84 | 101 |
| 90-Day | Uncapped | Pass | 4 | 78 | 82 |

### Example 5

A composition, according to an embodiment of the present invention, was prepared with the following amounts of ingredients:

| | | Proposed | |
|---|---|---|---|
| | | Composition | % |
| Ingredients | Function | (mg/unit) | Composition |
| Polyethylene Glycol 3350 USP | Active | 17000 | 84.794 |
| RM Sucralose NF | Sweetener | 17.5 | 0.087 |
| FONATECH ® CITRUS FLAVOR, | Flavor | 100 | 0.499 |
| NAT 865.0569U | | | |
| RM Monosodium Citrate F3500 | Effervescent | 1640 | 8.180 |
| | Agent | | |
| RM SOD BICARB #2 F-GRAN | Effervescent | 1291 | 6.439 |
| | Agent | | |
| Total (mg) | | 20048.5 | 100 |

We claim:

1. A powdered effervescent laxative composition, the powdered composition comprising:
about 70 weight percent or more of polyethylene glycol having a weight average molecular weight of between about 3,000 and about 4,000 g/mol;
an effervescent system including (i) an acid component selected from the group consisting of monosodium citrate, disodium citrate, trisodium citrate, maleic acid, fumaric acid, adipic acid, and combinations thereof and (ii) an alkaline component selected from the group consisting of sodium bicarbonate, sodium carbonate, potassium bicarbonate, potassium carbonate, calcium bicarbonate, calcium carbonate, and combinations thereof; and
wherein the effervescent system has a molar ratio of (i) the acid component to (ii) the alkaline component of about 1:1 to about 1:4.

2. The composition of claim 1, wherein the effervescent system is all-inclusive.

3. The composition of claim 1, which remains shelf-stable in multi-dose containers.

4. The composition of claim 1, wherein the molar ratio of (i) the acid component to (ii) the alkaline component is from about 1:1.5 to about 1:3.

5. The composition of claim 1, wherein the molar ratio of (i) the acid component to (ii) the alkaline component is from about 1:1.7 to about 1:3.

6. The composition of claim 1, wherein the molar ratio of (i) the acid component to (ii) the alkaline component is from about 1:1.9 to about 1:2.5.

7. The composition of claim 1, wherein the molar ratio of (i) the acid component to (ii) the alkaline component is about 1:2.

8. The composition of claim 1, wherein the alkaline component is selected from the group consisting of sodium bicarbonate, potassium bicarbonate, and calcium bicarbonate.

9. The composition of claim 8, wherein the alkaline component is sodium bicarbonate.

10. The composition of claim 1, wherein the alkaline component is selected from the group consisting of sodium carbonate, potassium carbonate, and calcium carbonate.

11. The composition of claim 10, wherein the ratio of (i) the acid component to (ii) the alkaline component is from about 1:1 to about 1:2.

12. The composition of claim 1, wherein the acid component comprises monosodium citrate.

13. The composition of claim 1, wherein the acid component comprises 50% or greater monosodium citrate by weight of the acid component.

14. The composition of claim 1, wherein the acid component comprises 90% or greater monosodium citrate by weight.

15. The composition of claim 1, wherein the effervescent system comprises citric acid in an amount of less than 5% by weight of the composition.

16. The composition of claim 1, wherein the effervescent system comprises citric acid, formic acid, acetic acid, oxalic acid or combinations thereof in an amount of less than about 0.5% by weight of the composition.

17. The composition of claim 1, wherein the polyethylene glycol is PEG 3350.

18. The composition of claim 1, comprising less than 30 ppm of formaldehyde after the composition is stored in a closed container at about 40° C. and 75% relative humidity for about 8 weeks.

19. The composition of claim 18, comprising less than 20 ppm of formaldehyde after the composition is stored in a closed container at about 40° C. and 75% relative humidity for about 8 weeks.

20. The composition of claim 1, comprising less than 200 ppm total aldehydes after the composition is stored in a closed container at about 40° C. and 75% relative humidity for about 8 weeks.

21. The composition of claim 1, wherein the acid component consists essentially of monosodium citrate.

22. The composition of claim 1, wherein the composition has a weight ratio of the polyethylene glycol to the (i) acid component of about 8:1 to about 15:1.

23. The composition of claim 22, wherein the composition has a weight ratio of the polyethylene glycol to the (i) acid component of about 9:1 to about 11:1.

24. The composition of claim 23, wherein the composition has a weight ratio of the polyethylene glycol to the (i) acid component of about 10:1.

25. The composition of claim 23, wherein the composition has a weight ratio of the polyethylene glycol to the (i) acid component of about 9:1.

26. The composition of claim 1, wherein the composition comprises about 75 to about 90 weight percent of the polyethylene glycol, about 4 to about 12 weight percent of the (i) acid component, and about 4 to about 10 weight percent of the (ii) alkaline component.

27. The composition of claim 26, wherein the composition comprises about 75 to about 90 weight percent of the polyethylene glycol, about 4 to about 12 weight percent of monosodium citrate, and about 4 to about 10 weight percent sodium bicarbonate.

28. The composition of claim 26, wherein the weight ratio of the polyethylene glycol to the acid component (i) is about 8.8:1 to about 12:1 and the molar ratio of (i) the acid component to (ii) the alkaline component is about 1:1.9 to about 1:2.5.

29. The composition of claim 1, wherein the composition remains shelf-stable for at least 6 months at 40° C. and 75% relative humidity.

30. The composition of claim 1, wherein the composition remains shelf-stable for at least 90 days when stored uncapped at 25° C. and 60% relative humidity.

31. The composition of claim 1, further comprising one or more of a flavoring agent, a sweetener, a colorant, or combinations thereof.

32. The composition of claim 31, wherein the sweetener is selected from the group consisting of glucose, sucrose, maltose, mannose, dextrose, fructose, lactose, trehalose, maltitol, lactitol, xylitol, sorbitol, mannitol, tagatose, glycerin, erythritol, isomalt, maltose, sucralose, aspartame, neotame, alitame, neohesperidin dihydrochalcone, cyclamate, thaumatin, acesulfame potassium, saccharin, saccharin sodium, and combinations thereof.

33. A packaged and storage stable powdered effervescent laxative composition, the packaged composition comprising:
a sealed container;
a powdered effervescent laxative composition contained in the sealed container; wherein the powdered effervescent laxative composition includes about 70 weight percent or more of polyethylene glycol having a weight average molecular weight of from 3000 to 4000 g/mol; an effervescent system including (i) an acid component selected from the group consisting of monosodium citrate, disodium citrate, trisodium citrate, maleic acid, fumaric acid, adipic acid, and combinations thereof and (ii) an alkaline component selected from the group consisting of sodium bicarbonate, sodium carbonate, potassium bicarbonate, potassium carbonate, calcium bicarbonate, calcium carbonate, and combinations thereof; and wherein the effervescent system has a molar ratio of (i) the acid component to (ii) the alkaline component of about 1:1 to about 1:4.

34. The packaged composition of claim 33, wherein the sealed container is a multi-dose or single-dose container.

35. The packaged composition of claim 34, wherein the sealed container is selected from the group consisting of a bottle, a sachet, a pouch, a vial, and a blister pack.

36. The packaged composition of claim 35, wherein the sealed container is a bottle having a cap and a seal.

37. The packaged composition of claim 36, wherein the bottle is formed of polyethylene (e.g., high density polyethylene), polypropylene, ethylene vinyl alcohol (EVOH), nylon, polyester, polyethylene terephthalate, glass, or combinations thereof.

38. The packaged composition of claim 35, wherein the container is a pouch having one or more heat seals.

39. The packaged composition of claim 38, wherein the pouch having one or more heat seals is formed of one or more layers selected from the group consisting of polyethylene, nylon, polyester, polypropylene, (meth)acrylate, (meth)acrylic acid, foil (e.g. aluminum foil), polyethylene terephthalate, metalized derivatives thereof, and combinations thereof.

40. The packaged composition of claim 33, wherein the sealed container is a multi-dose container which contains multiple doses of the powdered effervescent laxative composition.

41. The packaged composition of claim 33, wherein the acid component (i) consists essentially of monosodium citrate.

42. The packaged composition of claim 33, wherein the composition has a weight ratio of the polyethylene glycol to the acid component (i) of about 8:1 to about 15:1.

43. The packaged composition of claim 33, wherein the composition comprises about 75 to about 90 weight percent of the polyethylene glycol, about 4 to about 12 weight percent of the (i) acid component, and about 4 to about 10 weight percent of the (ii) alkaline component.

44. The packaged composition of claim 33, wherein the weight ratio of the polyethylene glycol to the acid component (i) is about 8.8:1 to about 12:1 and the molar ratio of (i) the acid component to (ii) the alkaline component is about 1:1.2 to about 1:1.5.

45. The packaged composition of claim 33, wherein the composition remains storage stable for at least 4 weeks at 40° C. and 75% relative humidity.

46. A powdered effervescent laxative composition, the powdered composition comprising:
about 70 weight percent or more of polyethylene glycol having a weight average molecular weight of between about 3,000 and about 4,000 g/mol;
an effervescent system including (i) an acid component selected from the group consisting of monosodium citrate, disodium citrate, trisodium citrate, maleic acid, fumaric acid, adipic acid, and combinations thereof and (ii) an alkaline component selected from the group consisting of sodium bicarbonate, sodium carbonate, potassium bicarbonate, potassium carbonate, calcium bicarbonate, calcium carbonate, and combinations thereof; and
wherein the effervescent system has a molar ratio of (i) the acid component to (ii) the alkaline component of about 1:1 to about 1:4;
wherein the product comprising less than 30 ppm of formaldehyde after storage in a closed container at about 40° C. and 75% relative humidity for about 8 weeks.

47. The powdered effervescent laxative composition of claim 46, wherein the molar ratio of (i) the acid component to (ii) the alkaline component is from about 1:1.5 to about 1:3.

48. The powdered effervescent laxative composition of claim 46, wherein the molar ratio of (i) the acid component to (ii) the alkaline component is from about 1:1.7 to about 1:3.

49. The powdered effervescent laxative composition of claim 46, wherein the molar ratio of (i) the acid component to (ii) the alkaline component is from about 1:1.9 to about 1:2.5.

50. The powdered effervescent laxative composition of claim 46, wherein the molar ratio of (i) the acid component to (ii) the alkaline component is about 1:2.

51. The powdered effervescent laxative composition of claim 46, wherein the alkaline component is selected from the group consisting of sodium bicarbonate, potassium bicarbonate, and calcium bicarbonate.

52. A powdered effervescent laxative composition, the powdered composition comprising:
PEG 3350 and an effervescent system including monosodium citrate and sodium bicarbonate;
wherein the composition comprises about 75 to about 90 weight percent of the PEG3350, about 4 to about 12 weight percent of the monosodium citrate, and about 4 to about 10 weight percent of the sodium bicarbonate; and
wherein the effervescent system has a molar ratio of monosodium citrate to sodium bicarbonate of from about 1:1.5 to about 1:3.

53. The composition of claim 52, wherein the effervescent system has a molar ratio of monosodium citrate to sodium bicarbonate of about 1:2.

54. The composition of claim 52, wherein the composition comprises from about 7 to about 10 weight percent of monosodium citrate and from about 5 to about 8 weight percent sodium bicarbonate.

55. The composition of claim 54, wherein the composition comprises from about 8 to about 9.5 weight percent of monosodium citrate and from about 6 to about 7.5 weight percent sodium bicarbonate.

56. A method of treating constipation in a subject in need thereof, comprising administering to the subject a composition, or a dose of a composition, of any one of claims 1-55.

57. A method of softening stool in a subject in need thereof, comprising administering to the subject a composition, or a dose of a composition, of any one of claims 1-55.

58. A method of increasing the frequency of bowel movements in a subject in need thereof, comprising administering to the subject a composition, or a dose of a composition, of any one of claims 1-55.

59. A method of relieving occasional constipation in a subject in need thereof, comprising administering to the subject a composition, or a dose of a composition, of any one of claims 1-55.
